(19) United States
(12) Patent Application Publication
(10) Pub. No.: US 2024/0398346 A1
(43) Pub. Date: Dec. 5, 2024

(54) WEARABLE VITAL SIGNS MONITOR AND USE THEREOF

(71) Applicant: Salyx Medical Inc., Victoria (CA)

(72) Inventors: Alfonso Fabian de la Fuente Sanchez, Victoria (CA); Sergio Perez Martell, Victoria (CA)

(73) Assignee: Salyx Medical Inc., Victoria (CA)

(21) Appl. No.: 18/325,851

(22) Filed: May 30, 2023

(51) Int. Cl. A61B 5/00; A61B 5/024; A61B 5/28

(52) U.S. Cl. CPC A61B 5/6833; A61B 5/02416; A61B 5/28; A61B 2562/0271; A61B 2562/164

(57) ABSTRACT

A vital signs monitoring system is provided, the vital signs monitoring system comprising a wearable patch and a mobile computing device, the wearable patch including: a flexible housing which includes a contact zone and a border zone around the contact zone; an adhesive on at least part of the border zone; an electrocardiogram embedded in the flexible housing; a photoplethysmogram embedded in the flexible housing; a temperature sensor embedded in the flexible housing, proximate to the contact zone and facing the contact zone; and a microprocessor which includes a wireless radio for transmitting data to the mobile computing device, the mobile computing device including: a memory; a processor; a wireless radio; and a screen, wherein the memory is configured to instruct the processor to convert the electrocardiogram data, the photoplethysmogram data and the temperature data into vital signs data and to display the vital signs data on the screen.

### FIELD

The present technology is directed to a wearable patch for the continuous monitoring of a user's temperature, pulse rate, heart rhythm, blood pressure, volumetric variations of blood circulation, oxygen saturation level, pulse rate, and respiratory rate. More specifically it is a vital signs monitoring system which includes a temperature sensor, an electrocardiogram (ECG) sensor, photoplethysmogram (PPG) sensor and an application on a mobile computing device.

### BACKGROUND

United States Patent Application Publication No. 20230095971 discloses a finger wearable device for monitoring vital signs and includes a housing, a finger cuff, a plurality of vital sign sensors, and an electrocardiogram (ECG) sensor. The housing includes an interface surface for pressing against the finger. The finger cuff attaches to the housing and has a size and a shape to secure the housing to the finger and force the interface surface against the finger when the finger cuff is worn around the finger. The vital sign sensors are disposed in or on the housing and orientated to measure the vital signs from the finger of a wearer. The ECG sensor is disposed in or on the housing and coupled to first and second electrodes to measure ECG signals. The second electrode is disposed on the interface surface. This is a very complex system for which the accuracy is not known.

United States Patent Application Publication No. 20180303419 discloses a wearable vital sign monitor. The wearable vital sign monitor includes a band, where the band secures the wearable vital sign monitor relative to a user. The band includes an interior surface, the interior surface being the surface closest to the skin of the user and an exterior surface, the exterior surface opposite the interior surface. The wearable vital sign monitor also includes a first sensor array. The first sensor array is attached to the band and includes at least two sensors. The wearable vital sign monitor further includes a second sensor array. The second sensor array is attached to the band a fixed distance from the first sensor array and includes at least two sensors. The wearable vital sign monitor additionally includes an electronics module. The electronics module is configured to receive a first signal from the first sensor array, receive a second signal from the second sensor array, and transmit the sensor data to an external device. It is not known how accurate the sensors are.

U.S. Pat. No. 11,109,765 discloses a wearable thermometer patch for monitoring healing of a wound on a user's skin includes a circuit substrate comprising an electric circuit and a plurality of temperature probe units distributed in a two-dimensional array. Each of the plurality of temperature probe units can include a temperature sensor in electric connection with the electric circuit in the circuit substrate. The plurality of temperature probe units can measure temperature values on a user's skin at different positions defined by the two-dimensional array. This would be uncomfortable for the user as the temperature probes protrude.

U.S. Pat. No. 10,420,473 discloses a wearable thermometer patch that includes a substrate and a temperature probe unit mounted in the substrate and configured to measure temperature of a user's skin. The temperature probe unit includes a force sensor configured to measure contact force between the temperature probe unit and the user' skin, a plate, a first temperature sensor attached to a lower surface of the plate, and a second temperature sensor attached to an upper surface of the plate.

What is needed is a small, wearable vital signs monitor. It would be preferable if it was a patch that could be worn by a user. It will be preferable if the patch had an ECG sensor, a PPG sensor and a thermistor embedded therein. It would be preferable if the vital signs monitor was provided as a system that included an application on a mobile computing device. It would be preferable if the vital signs monitor included a wireless radio for communicating data to the application. It would also be preferable if the application included a machine learning component that could enhance the monitoring capabilities of the device.

### SUMMARY

The present technology is a small, wearable vital signs monitor. It is a patch that can be worn by a user. The patch has an ECG sensor, a PPG sensor and a thermistor embedded therein. The vital signs monitor is provided as a system that includes an application on a mobile computing device. The vital signs monitor includes a wireless radio for communicating data to the application. The application includes a machine learning component to enhance the monitoring capabilities of the device.

In one embodiment, a vital signs monitoring system is provided, comprising a wireless wearable patch and a mobile computing device. The wearable patch consists of a flexible housing, a thermally conductive material inside a contact zone, and a border region around the contact zone, an adhesive on at least part of the border region. Within the flexible housing there is an electrocardiogram sensor, which includes a first electrode on the border region and a second electrode for releasable attachment on a user at a site remote to the wearable patch or is on the border region; a photoplethysmogram sensor embedded in the flexible housing; a temperature sensor embedded in the flexible housing, at to the contact zone and facing the contact zone; and a microprocessor which includes a wireless radio for transmitting data to the mobile computing device. The mobile computing device including: a memory; a processor; a wireless radio; and a screen, wherein the memory is configured for machine learning and to instruct the processor to convert the electrocardiogram data, the photoplethysmogram data and the temperature data into vital signs data and to display the vital signs data on the screen.

In the vital signs monitoring system, the flexible housing may be comprised of silicon material.

In the vital signs monitoring system, the contact zone may be a silicon thermal pad or a graphite thermal pad.

In the vital signs monitoring system, the temperature sensor may be a thermistor.

In another embodiment, a wearable vital signs monitoring patch is provided for use with a mobile computing device, the wearable vital signs monitoring patch comprising: a flexible housing which includes a contact zone, which is a thermally conductive material and a border region around the contact zone; an adhesive on at least part of the border region; an electrocardiogram sensor embedded in the flexible housing; a photoplethysmogram sensor embedded in the flexible housing; a temperature sensor embedded in the flexible housing and located at the contact zone and facing the contact zone; and a microprocessor which includes a wireless radio for transmitting data to the mobile computing device.

In the vital signs monitoring system, the flexible housing may be comprised of silicon material.

In the wearable vital signs monitoring patch, the electrocardiogram sensor may include two electrodes, one of which is on the border region and one which is for locating on a user at a location remote to the wearable vital signs monitoring patch.

In the wearable vital signs monitoring patch, the electrocardiogram sensor may include two electrodes, both of which are on the border region.

In the wearable vital signs monitoring patch, the thermally conductive material may be a silicon thermal pad or a graphite thermal pad.

In another embodiment, a method of monitoring a user's vital signs is provided, the method comprising: selecting the vital signs monitoring system described above; the user releasably attaching the wearable patch to a selected skin surface; the user releasably attaching the second electrocardiogram sensor electrode to a selected skin surface remote to the wearable patch; the electrocardiogram sensor sending and receiving electrical signals, the photoplethysmogram sensor sending and receiving light signals, and the temperature sensor sensing the user's temperature, all to provide a data set; the wireless radio sending the data set to the mobile computing device; the mobile computing device analyzing the data set to provide a vital signs reading; and the mobile computing device displaying the vital signs reading.

In the method, displaying the vital signs reading may be in real time.

In another embodiment, a vital signs monitoring system is provided, the vital signs monitoring system comprising a wearable patch, a mobile computing device and a remote computer, the wearable patch including: a flexible housing which includes a contact zone, which is a thermally conductive material and a border region around the contact zone; an adhesive on at least part of the border zone; an electrocardiogram sensor embedded in the flexible housing; a photoplethysmogram sensor embedded in the flexible housing; a temperature sensor embedded in the flexible housing, proximate to the contact zone and facing the contact zone; and a microprocessor which includes a wireless radio for transmitting data to the mobile computing device, the mobile computing device including: a memory; a processor; a wireless radio; and a screen, and the remote computer including: a memory; a processor; and a wireless radio, wherein the memory is configured for machine learning and to convert the electrocardiogram data, the photoplethysmogram data and the temperature sensor data into vital signs data and send the vital signs data to the mobile computing device.

In the vital signs monitoring system, the flexible housing may comprise silicon.

In the vital signs monitoring system, the thermally conductive material may be a silicon thermal pad or a graphite thermal pad.

In the vital signs monitoring system, the electrocardiogram sensor may include two electrodes, of which one is on the border region and one which is configured for placement on a user's skin remote to the wearable patch.

In another embodiment, a method of monitoring a user's vital signs is provided, the method comprising: selecting the vital signs monitoring system as described above; the user releasably attaching the wearable patch to a selected skin surface; the user releasably attaching one electrode at a location on the user remote to the wearable patch; the electrocardiogram sensor sending and receiving electrical signals, the photoplethysmogram sensor sending and receiving light signals and the temperature sensor sensing the user's temperature, all to provide a data set; the wireless radio sending the data set to the mobile computing device; the mobile computing device sending the data set to the remote computer; the remote computer analyzing the data set to provide a vital signs reading; the remote computer sending the vital signs reading to the mobile computing device; and the mobile computing device displaying the vital signs reading on the screen.

In the method, displaying the vital signs reading may be in real time.

### BRIEF DESCRIPTION OF THE DRAWINGS

FIG. 1 is a schematic of the vital signs monitoring system of the present technology.

FIG. 2A is a schematic of a sectional view of the vital signs monitoring patch; and FIG. 2B is a schematic of a sectional view of an alternative embodiment of the vitals signs monitoring system.

FIG. 3 is a schematic of the machine learning system of the present technology.

FIG. 4 is a flow chart of the learning process.

FIG. 5 is a flow chart of the method of blood pressure monitoring of the present technology.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Mobile computing device—in the context of the present technology a mobile computing device is a cellular phone, a smart phone, a tablet, a laptop, wearable computing device, personal digital assistant, mobile Internet device, notebook, netbook, smart device and any computing device that is either hand-held or can easily be held by an individual.

Computing device—in the context of the present technology, a computing device includes at least one processor, a network adapter, and computer-readable storage media. A network adapter may be any suitable hardware and/or software to enable the computing device to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media may be adapted to store data to be processed and/or instructions to be executed by processor. The processor enables processing of data and execution of instructions. The data and instructions may be stored on the computer-readable storage media.

A computing device may additionally have one or more components and peripherals, including input and output devices. Examples of output devices that can be used to provide a user interface or display screens for visual presentation of output and speakers or other sound generating devices for audible presentation of output.

Computer executable instructions—in the context of the present technology, computer executable instructions include software, including as application software, system software, firmware, middleware, embedded code, or any other suitable type of computer code and also may be compiled as executable machine language code or intermediate code.

Communication network—in the context of the present technology a communication network includes, but is not limited to a wireless fidelity (Wi-Fi [IEEE 802.11]) network, a light fidelity (Li-Fi) network, a satellite network, the internet, a cellular data network, a local area network (LAN), a wireless local area network (WLAN), or any combination thereof. The network adapter of the computing device communicates via the communication network.

### DETAILED DESCRIPTION

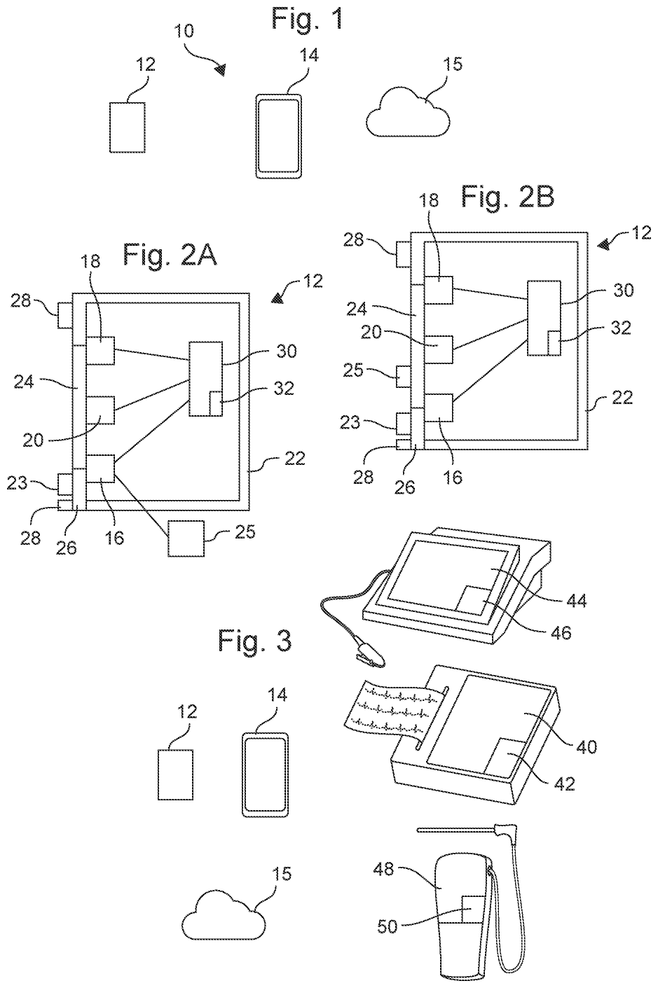

A vital signs monitoring system, generally referred to as 10 is shown in FIG. 1. The vital signs monitoring system 10 includes a wearable patch 12 and a mobile computing device 14. In one embodiment, the system 10 includes a remote computer 15 which provides cloud processing.

As shown in FIG. 2A, the wearable patch 12 has an electrocardiogram (ECG) sensor 16, a photoplethysmogram (PPG) sensor 18 and a temperature sensor 20 that are embedded in a flexible housing 22. The ECG sensor 16 has two electrodes 23, 25, one which is on a border region 26 of the flexible housing 22. The second electrode is for locating on the user in a position remote to the wearable patch 12. The temperature sensor 20 is facing and proximate to the contact zone 24, which is a thermally conductive material, for example, but not limited to a silicon thermal pad, a graphite thermal pad, a copper pad or an aluminum pad. In one embodiment the remainder of the flexible housing 22 is silicon. In one embodiment the temperature sensor is a thermistor. The border region 26, which surrounds the contact zone 24 is coated, at least partially, with an adhesive layer 28, for affixing the wearable patch 12 to a user. The wearable patch 12 is small and is preferably affixed to a user's armpit. The ECG sensor 16, the PPG sensor 18 and the temperature sensor 20 are in electrical communication with at least one microcontroller 30. The microcontroller 30 includes a wireless radio 32. An electrical system, which includes a battery, is in electrical communication with the sensors and the microcontroller 30.

An alternative embodiment is shown in FIG. 2B. The wearable patch 12 has an electrocardiogram (ECG) sensor 16, a photoplethysmogram (PPG) sensor 18 and a temperature sensor 20 that are embedded in a flexible housing 22. The ECG sensor 16 has two electrodes 23, 25, both which are on a border region 26 of the flexible housing 22 at opposite ends of the border region 26. The temperature sensor 20 is facing and proximate to the contact zone 24, which is a thermally conductive material, for example, but not limited to a silicon thermal pad, a graphite thermal pad, a copper pad or an aluminum pad. In one embodiment the remainder of the flexible housing 22 is silicon. In one embodiment the temperature sensor is a thermistor. The border region 26, which surrounds the contact zone 24 is coated, at least partially, with an adhesive layer 28, for affixing the wearable patch 12 to a user. The wearable patch 12 is small and is preferably affixed to a user's armpit. The ECG sensor 16, the PPG sensor 18 and the temperature sensor 20 are in electrical communication with at least one microcontroller 30. The microcontroller 30 includes a wireless radio 32. An electrical system, which includes a battery, is in electrical communication with the sensors and the microcontroller 30.

As shown in FIG. 3, a machine learning system includes the wearable patch 12, the mobile computing device 14, an ECG machine 40, which includes a wireless radio 42, a PPG machine 44, which includes a wireless radio 46 and electronic thermometer 48, which include a wireless radio 50. In an alternative embodiment, the wireless radios 42, 46 and 50 are replaced with wireless transmitters. In another alternative embodiment, remote computer 15 is part of the machine learning system.

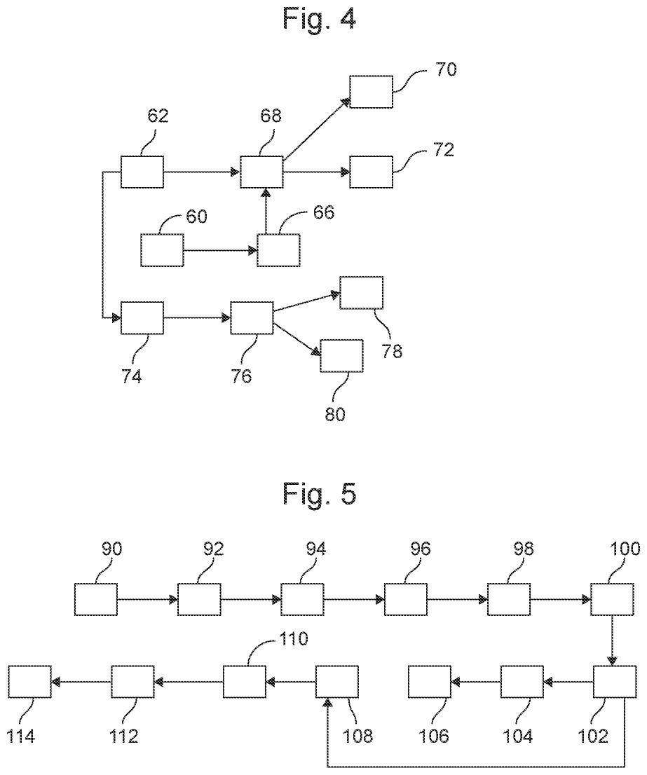

As shown in FIG. 4, the memory of the mobile computing device or the memory of the remote computer is configured for learning. During the learning phase, the ECG machine 40, the PPG machine and the electronic thermometer 48 concomitantly measure 60 temperature, heart rate, heart rhythm, blood pressure, volumetric variations of blood circulation, oxygen saturation level, pulse rate, and respiratory rate as the wearable patch 12 is collecting 62 temperature, heart rate, heart rhythm, blood pressure, volumetric variations of blood circulation, oxygen saturation level, pulse rate, and respiratory rate. The wireless radios 42, 46, 50 send 66 the data to the mobile computing device 14. The two data sets are compared 68 and data from the wearable device 12 that do not agree with the data from the ECG machine 40, the PPG machine 44 and the electronic thermometer 48 are discarded 70. The process is repeated until the memory has a large enough set of valid data to be considered trained 72. In an alternative embodiment, the mobile computing device sends 74 the data to the remote computer. The two data sets are compared 76 and data from the wearable device 12 that do not agree with the data from the ECG machine 40, the PPG machine 44 and the electronic thermometer 48 are discarded 78. The process is repeated until the memory has a large enough set of valid data to be considered trained 80.

As shown in FIG. 5, a user releasably attaches 90 the wearable patch 12 to their skin, preferably on their arm. The user opens 92 the application on their mobile computing device 14 and clicks 94 on “start”. The mobile computing device 14 communicates 96 wirelessly to the microprocessor 24 to tell it to instruct the electrocardiogram sensor 16, the photoplethysmogram sensor 18 and the temperature sensor 20 to function. The microprocessor instructs 98 the electrocardiogram sensor 16 to emit and receive electrical signals, the photoplethysmogram sensor 18 to emit and receive light and the temperature sensor 20 to measure temperature. Each of the electrocardiogram sensor 16, the photoplethysmogram sensor 18 and the temperature sensor 20 record 100 data. The data are then sent 102 wirelessly via the wireless radio 26 to the mobile computing device 14. The memory is configured to analyze the data to determine the user's temperature, heart rate, heart rhythm, blood pressure, volumetric variations of blood circulation, oxygen saturation level, pulse rate, and respiratory rate, hence the data are analyzed 104 and the results are displayed 106 on the screen of the mobile computing device 14 in real time. In an alternative embodiment, the mobile computing device sends 108 the data wirelessly to the remote computer 15. The memory of the remote computer 15 is configured to analyze the data to determine the user's temperature, heart rate, heart rhythm, blood pressure, volumetric variations of blood circulation oxygen saturation level, pulse rate, and respiratory rate hence the data are analyzed 110, then sent 112 back to the mobile computing device 14 and the results are displayed 114 on the screen of the mobile computing device 14 in real time.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A vital signs monitoring system, the vital signs monitoring system comprising a wearable patch and a mobile computing device, the wearable patch including: a flexible housing which includes a contact zone, which is a thermally conductive material and a border region around the contact zone; an adhesive on at least part of the border region; an electrocardiogram sensor embedded in the flexible housing, wherein the electrocardiogram sensor includes a first electrode which is on the border region and a second electrode which is either on the border region or is for releasable attachment on a user at a site remote to the wearable patch; a photoplethysmogram sensor embedded in the flexible housing; a temperature sensor embedded in the flexible housing, at to the contact zone and facing the contact zone; and a microprocessor which includes a wireless radio for transmitting data to the mobile computing device, the mobile computing device including: a memory; a processor; a wireless radio; and a screen, wherein the memory is configured for machine learning and to instruct the processor to convert the electrocardiogram data, the photoplethysmogram data and the temperature data into vital signs data and to display the vital signs data on the screen.

2. The vital signs monitoring system of claim 1, wherein the flexible housing comprises silicon.

3. The vital signs monitoring system of claim 2, wherein the contact zone is a silicon thermal pad or a graphite thermal pad.

4. The vital signs monitoring system of claim 3, wherein the temperature sensor is a thermistor.

5. The vital signs monitoring system of claim 4, wherein the second electrode is on the border region.

6. A wearable vital signs monitoring patch for use with a mobile computing device, the wearable vital signs monitoring patch comprising: a flexible housing which includes a contact zone, which is a thermally conductive material and a border region around the contact zone; an adhesive on at least part of the border region; an electrocardiogram sensor embedded in the flexible housing, wherein the electrocardiogram sensor includes a first electrode which is on the border region and a second electrode which is either on the border region or is for releasable attachment on a user at a site remote to the wearable patch; a photoplethysmogram sensor embedded in the flexible housing; a temperature sensor embedded in the flexible housing, located at the contact zone and facing the contact zone; and a microprocessor which includes a wireless radio for transmitting data to the mobile computing device.

7. The wearable vital signs monitoring patch of claim 6, wherein the flexible housing comprises silicon.

8. The wearable vital signs monitoring patch of claim 7, wherein the thermally conductive material is a silicon thermal pad or a graphite thermal pad.

9. The wearable vital signs monitoring patch of claim 8, wherein the second electrode is on the border region.

10. A method of monitoring a user's vital signs, the method comprising: a user selecting the vital signs monitoring system of claim 5; the user providing a power source to the wearable patch; the user releasably attaching the wearable patch to a selected skin surface; the electrocardiogram sensor sending and receiving electrical signals, the photoplethysmogram sensor sending and receiving light signals, and the temperature sensor sensing the user's temperature, all to provide a data set; the wireless radio sending the data set to the mobile computing device; the mobile computing device analyzing the data set to provide a vital signs reading; and the mobile computing device displaying the vital signs reading.

11. The method of claim 10 wherein the displaying the vital signs reading is in real time.

12. A vital signs monitoring system, the vital signs monitoring system comprising a wearable patch, a mobile computing device and a remote computer, the wearable patch including: a flexible housing which includes a contact zone, which is a thermally conductive material and a border region around the contact zone; an adhesive on at least part of the border zone; an electrocardiogram sensor embedded in the flexible housing; a photoplethysmogram sensor embedded in the flexible housing; a temperature sensor embedded in the flexible housing, proximate to the contact zone and facing the contact zone; and a microprocessor which includes a wireless radio for transmitting data to the mobile computing device, the mobile computing device including: a memory; a processor; a wireless radio; and a screen, and the remote computer including: a memory; a processor; and a wireless radio, wherein the memory is configured for machine learning and to convert the electrocardiogram data, the photoplethysmogram data and the temperature sensor data into vital signs data and send the vital signs data to the mobile computing device.

13. The vital signs monitoring system of claim 12, wherein the flexible housing comprises silicon.

14. The vital signs monitoring system of claim 13, wherein the thermally conductive material is a silicon thermal pad or a graphite thermal pad.

15. The vital signs monitoring system of claim 14, wherein the electrocardiogram sensor includes two electrodes, of which one is on the border region and one which is configured for placement on a user's skin remote to the wearable patch.

16. The vital signs monitoring system of claim 14, wherein the second electrode is on the border region.

17. A method of monitoring a user's vital signs, the method comprising: a user selecting the vital signs monitoring system of claim 16; the user providing a power source to the wearable patch; the user releasably attaching the wearable patch to a selected skin surface; the electrocardiogram sensor sending and receiving electrical signals, the photoplethysmogram sensor sending and receiving light signals and the temperature sensor sensing the user's temperature, all to provide a data set; the wireless radio sending the data set to the mobile computing device; the mobile computing device sending the data set to the remote computer; the remote computer analyzing the data set to provide a vital signs reading; the remote computer sending the vital signs reading to the mobile computing device; and the mobile computing device displaying the vital signs reading on the screen.

18. The method of claim 17, wherein the displaying the vital signs reading is in real time.
